(19) United States
(12) Patent Application Publication
(10) Pub. No.: US 2021/0274816 A1
(43) Pub. Date: Sep. 9, 2021

(54) REFRESHMENT DRINK

(71) Applicant: HeraVita GmbH, Dortmund (DE)

(72) Inventors: Bani ETEMAD, Dortmund (DE); Mani ETEMAD, Dortmund (DE); Shahram ETEMAD, Dortmund (DE)

(21) Appl. No.: 16/336,666

(22) PCT Filed: Oct. 7, 2017
(86) PCT No.: PCT/EP2017/075491

§ 371 Date: Mar. 26, 2019

(30) Foreign Application Priority Data
Oct. 6, 2017 (EP) EP2017075491

(51) Int. Cl. A23L 2/68; A23L 2/54; A23L 33/105

(52) U.S. Cl. CPC A23L 2/68; A23L 33/105; A23L 2/54

(57) ABSTRACT

The invention relates to a refreshment drink which contains a spice dissolved in water, and further additives as appropriate. According to the invention, the water contains CO₂.

The invention relates to a soft drink containing a herb dissolved in water and, optionally, further additives.

Soft drinks with a herb dissolved in water as a remedy are known. For instance, U.S. Pat. No. 6,355,279 describes a beverage composition with the aid of which the metabolism, i.e. the metabolism in humans, is to be improved. In this context, various herb-based liquid remedies are being investigated, including those based on turmeric.

Turmeric is a known plant of the ginger family that is used as a herb plant and as a dye for yellow in the coloring of foods. In addition, the positive curative effect of such herbs for reducing triglyceride levels in the blood has been investigated and demonstrated in the framework of U.S. Pat. No. 6,355,279. Elevated triglyceride levels in the blood are in fact often indicative of a lipid metabolism disorder or excess weight. Moreover, they are is associated with diseases such as arteriosclerosis or kidney diseases that are also associated with an elevated cholesterol level. In any case, the positive and curative effect of such herb mixtures on human health becomes clear from the explanations provided in U.S. Pat. No. 6,355,279. This is especially true with regard to the reduction of fatty substances dissolved in the blood and hence also a reduced probability of corresponding diseases with appropriate intake.

U.S. Pat. No. 6,355,279 describes the soft drink in question as a medicinal drink, as well as the preparation thereof. As usual, the concentration of the dissolved herb is relatively high in the medicinal drink in question, so that further additives such as honey or citric acid are ultimately required as herb additives in order to ensure palatability. As a result, such medicinal drinks are often very expensive and are usually sold only through pharmacies or drugstores. What is more, their intense taste often hampers wide and desirable distribution.

The object of the invention is therefore to further develop such a soft drink in such a way that broad acceptance is achieved at costs that are lower compared to the prior art.

To attain this object, a soft drink of this generic type is characterized in that the water contains CO₂.

Therefore, what is achieved in the context of the invention is, first of all, a soft drink based on a CO₂-containing water in which a herb is dissolved. According to an advantageous embodiment, the herb in the CO₂-containing water in question is dissolved in a concentration of no more than 80 g, particularly 50 g, preferably no more than 20 g, and especially preferably no more than 10 g/l (grams per liter) CO₂-containing water. The indicated quantity refers to the anhydrous mass of the corresponding herb.

In principle, the CO₂-containing water can be a carbonated tap water, i.e. an artificially produced soft drink. In fact, tap water consists of drinking water that is enriched with other ingredients. In the context of the invention, the tap water in question is enriched with CO₂, or carbon dioxide. In principle, a so-called soda water, i.e. a tap or mineral water in which at least 570 mg of sodium bicarbonate (soda) are additionally dissolved per liter, can also be used as a CO₂-containing water. However, a natural CO₂-containing mineral water can also be used as the basis for the soft drink according to the invention. Mixtures are also conceivable.

The natural mineral water can be one that is mixed with a spring carbonic acid or also a natural mineral water that is (additionally) mixed with carbonic acid. In any case, the CO₂-containing water used in the soft drink according to the invention generally has a CO₂ content in the water that can be routinely up to 10 g/l. Concentrations of CO₂ in the water of up to 7 g/l and, more preferably, up to 6 g/l are preferably observed. In principle, however, the CO₂ content in the water can also be below 250 mg/l. However, higher carbon dioxide levels are also possible and included.

Moreover, it has proven advantageous if the soft drink according to the invention is provided in chilled form for consumption, i.e. ready to drink. Temperatures in the range between 5° C. and 15° C. have proven to be especially favorable here. An overall tasty, refreshing drink is thus provided that produces a special curative effect as a result of the herb or plurality of herbs as well as the optional other additives dissolved in the water.

In fact, with regular consumption of the soft drink according to the invention, the level of fats or fatty acids in the blood in particular should be reduced as has already been demonstrated in the prior art for example according to U.S. Pat. No. 6,355,279. As a consequence of this, associated clinical issues such as infarction and stroke as well as diseases associated with blood-lipid deposits such as dementia are reduced, and a lower risk of the occurrence of such diseases is to be expected. All in all, this curative effect is achieved with a tasty soft drink previously considered to be impossible in this combination and unknown.

Moreover, inflammation reduction is observed as a further special effect that is inherently known in the use of herbs such as turmeric, ginger, rosemary, and cloves. Reference is made in this regard to the publication “Bioavailability of herbs and herbs in humans as determined by ex vivo inflammatory suppression and DNA strand breaks” (Journal of American Coll. Nutr., 2012 August; 31(4):288-94). Up to now, however, these effects had been observed in association with tablets or other dosage forms and not in the context of a soft drink.

Additional advantageous embodiments will be described below. The herb is typically present in a minimum concentration of 0.1 g/l of CO₂-containing water and is dissolved therein. In principle, however, a higher minimum concentration can also be used. In this case, the herb is dissolved in the CO₂-containing water in a concentration of at least 1 g and preferably at least 2 g/l of CO₂-containing water. Again, the indicated quantities are based on the anhydrous mass of the herb. The previously mentioned upper limit of 80 g of the herb/l of CO₂-containing water and preferably 50 g, especially preferably 20 g, and most preferably 10 g/l of CO₂-containing water are observed. Moreover, according to an advantageous embodiment, the herb is dissolved in the CO₂-containing water in question in a concentration of from 0.1 g to 20 g and preferably from 0.1 g to 10 g/l of CO₂-containing water. These indicated quantities are also based on the anhydrous mass of herb.

With regard to the addition of the herb to the CO₂-containing water, it has been found to be expedient to dissolve it in the CO₂-containing water in ground form. This enables the herb in question to be handled and added to the CO₂-containing water in the desired concentration with particular ease. In principle and alternatively, however, the herb can also be added as an extract to the CO₂-containing water. In this case, the herb may be available as an extract dissolved in water. Distilled water can be used to prepare the extract, for example. Additional heating of the extract to 80° C. or more, for example, for a period of 30 minutes, for example, is also possible.

In principle, the above-described extract of the herb can also be a juice or extraction or even a tea containing the herb in question. In general, the extract can be added to the CO₂-containing water in an amount of between 1 ml and 200 ml, preferably between 1 ml and 150 ml, and most preferably between 1 ml and 100 ml of extract per liter of CO₂-containing water. In the soft drink produced in this way, the herb continues to be present without change as an extract (juice, infusion, tea, or the like) in the above-described concentration of between 0.1 g as the minimum and 80 g as the maximum in 1 l of CO₂-containing water.

As will readily be understood, the other concentrations mentioned above can also be realized with the aid of such an extract. All of the stated values of, for example, 0.1 g as the minimum and 80 g as the maximum for the concentration of the herb/l of CO₂-containing water refer to the anhydrous mass of the herb in each case. This also applies to a case in which the herb is made by for example pressing from a tuber.

As a rule, however, the herb to be added to the CO₂-containing water is present in ground form, and hence as a powder. As a result, natural mineral water with its own spring carbonic acid, for example, that is being used as the basis can be mixed directly with the powder in question, so that the soft drink is then immediately available for transport and consumption.

The herb used according to the invention can be one from the group of turmeric, moringa, cinnamon, saffron, caraway, anise, mint, ginger, cloves, rosemary, vanilla, and cardamom, individually or in combination. The use of turmeric is very especially preferred in this context, with cinnamon and/or anise also being added as a further herb. In any case, the proportion of the herb in the CO₂-containing water is no more than 80 g/l and at least 0.1 g/l, with the weight references being respectively based on the anhydrous mass of the herb.

Fruits, aromas, and/or foodstuffs can also be given consideration as further optional additives. As a rule, the above-described constituents are present individually or in combination as additives. This is generally performed with a grammage for the additive of from 0 to 5 g and particularly from 0 to 3 g/l if the additive is in solid form and added to the CO₂-containing water. For example, this may apply to a fruit additive such as the chili pepper plant that, like the herb, is added to the CO₂-containing water in powdered form.

However, liquid additives are also conceivable, for example aromas such as rose water. Rose water is an essential water of the rose that is a byproduct of the distillation of rose oil from rose petals. Rose water is typically used in the food industry for aromatization. In this case, the invention recommends the addition of such liquid flavors such as rose water in an amount of from 0 to 200 ml per one liter of CO₂-containing water. Other aromas can of course also be added in this context.

Moreover, it lies within the scope of the invention to work with a food as an additive as an alternative or in addition. Taste-intensive foods such as honey have proven to be especially expedient here. In this case as well, the food in question is typically added to the CO₂-containing water in liquid form. In fact, the invention recommends an amount in the range of from 0 to 800 ml/l of CO₂-containing water. An amount in the range of from 0 to 200 ml/l of CO₂-containing water is preferably used for the additive.

That is, the liquid additives are generally present in a (cumulative) amount of from 0 to 200 ml per 11 liter of CO₂-containing water. Preferably, amounts of liquid in the range of from 10 ml to 100 ml/l of CO₂-containing water are observed and adjusted. If a plurality of liquid additives are used, their total amount should not exceed a total quantity of 200 ml per one liter of CO₂-containing water. In the event that additives in the form of solids or powders, such as chili powder, are given consideration in the exemplary case, the invention recommends an upper limit of 10 g and preferably 5 g in the sum per one liter for the powdery additives in question.

As a result, a refreshing drink is provided that is not only delicious, but also has or develops a health-promoting effect at the same time due to the addition of the one or more herbs. The CO₂-containing water used here, more particularly the carbon dioxide dissolved therein, stimulates the consumer's taste buds when drinking, so that the desired refreshing effect is observed. This is further supported by the fact that the soft drink according to the invention is generally served in ready-to-drink chilled form.

What is more, through the addition of one or more herbs, particularly turmeric, a health-promoting effect is observed with regular consumption. In fact, turmeric is characterized in that fats dissolved in the blood are reduced, so that the metabolism is stimulated overall and secondary diseases due to increased blood lipids are substantially reduced in frequency with regular consumption. At the same time, anti-inflammatory effects are observed. In addition, phenomena such as morning sickness or motion sickness, for example, can be controlled and alleviated with the soft drink according to the invention. Likewise, a positive effect on migraine is observed. Besides lowering the blood lipid content, a reduction of the blood sugar level is also observed. Herein lie the fundamental advantages.

1-13. (canceled)

14. A soft drink comprising:
water containing CO₂, and
an herb dissolved or suspended in the water.

15. The soft drink defined in claim 14, wherein a CO₂ content in the water is up to 10 g/l.

16. The soft drink defined in claim 15 wherein the CO₂ content in the water is below 250 mg/l.

17. The soft drink defined in claim 14, wherein the herb is dissolved in a concentration of no more than 80 g/l.

18. The soft drink defined in claim 17, wherein the herb is dissolved in a concentration of no more than 10 g/l of CO₂-containing water.

19. The soft drink defined in claim 14, wherein the herb is dissolved in a concentration of at least 1 g/l of CO₂-containing water.

20. The soft drink defined in claim 19, wherein the herb is dissolved in a concentration of at least 2 g/l of CO₂-containing water.

21. The soft drink defined in claim 14, wherein the herb is dissolved in a concentration of 0.1 g to 20 g/l of CO₂-containing water.

22. The soft drink defined in claim 14, wherein the herb is dissolved in a concentration of 0.1 g to 10 g/l of CO₂-containing water.

23. The soft drink defined in claim 14, wherein the herb is dissolved in ground form in the CO₂-containing water.

24. The soft drink defined in claim 14, wherein the herb is added as a dissolved extract to the CO₂-containing water.

25. The soft drink defined in claim 14, wherein the herb is turmeric, moringa, cinnamon, saffron, caraway, anise, mint, cloves, rosemary, vanilla, or cardamom or combinations thereof.

26. The soft drink defined in claim 14, further comprising:
an additive comprised of fruits, aromas, or foodstuffs.

27. The soft drink defined in claim 26 wherein the additive is added in a solid form and in an amount of from 0 to 5 g/l of CO₂-containing water.

28. The soft drink defined in claim 26, wherein the additive is added in liquid form to the CO₂-containing water in a concentration of from 0 to 800 ml/l.

29. The soft drink defined in claim 28, wherein the concentration of additive is from 0 to 200 ml/l CO₂-containing water.

30. The soft drink defined in claim 14, wherein the CO₂-containing water is tap water, soda water, or natural mineral water.

31. The soft drink defined in claim 14, wherein the drink is ready to drink in a cooled form at a temperature between 5° C. and 15° C.
